(19) United States
(12) Patent Application Publication
(10) Pub. No.: US 2001/0020104 A1
(43) Pub. Date: Sep. 6, 2001

(54) Process for preparing substituted benzyl compounds and toluene derivatives

(75) Inventors: Steffen Haber, Landau/Pfalz (DE); Stefan Scherer, Buttelborn (DE); Heinz Georg Kautz, Bierstein (DE); Andreas Meudt, Florsheim-Weilbach (DE); Frank Vollmuller, Frankfurt am Main (DE)

Correspondence Address: CLARIANT CORPORATION, 4331 CHESAPEAKE DR, ATTN: INDUSTRIAL PROPERTY DEPT, CHARLOTTE, NC, 28216, US

(73) Assignee: Clariant GmbH

(21) Appl. No.: 09/790,757

(22) Filed: Feb. 22, 2001

(30) Foreign Application Priority Data
Feb. 22, 2000 (DE) 10007939.3

(51) Int. Cl. C07C 2/02; C07C 6/12

(52) U.S. Cl. 549/434; 585/422

(57) ABSTRACT

The novel two-stage process described here makes it possible to obtain substituted benzyl compounds and toluene derivatives in a simple manner and in high yields by means of Suzuki-type coupling reactions of an aromatic with an organoboron compound, followed by a reduction. The process is particularly useful for preparing ortho-substituted benzyl compounds and toluene derivatives. The process can be applied to both intermolecular and intramolecular coupling reactions. Catalysts used for the coupling reaction are palladium compounds and/or nickel compounds. An advantageous aspect is that only very small amounts of catalyst are required.

### BACKGROUND OF THE INVENTION

Benzyl compounds and toluene derivatives having complex substitution can often be prepared by relatively old, established processes, for example Gomberg-Bachmann or Ullmann couplings, only if low yields or complicated purification processes are accepted. This is particularly true of unsymmetrically substituted aromatics.

In the past twenty years, numerous synthetic methods based on the coupling of two suitable substrates in the presence of transition-metal catalysts have been developed. An example is Suzuki-type couplings (Chem. Rev. 1995, 95, 2457; Tetrahedron Lett. 1979, 3437), in which an organoboron compound is coupled with a substrate bearing a potential leaving group using transition metals, generally palladium or nickel compounds, in the presence of a base.

Suitable leaving groups are the most frequently used halogens (I, Br, Cl, F) and sulfonates (e.g. —OSO₂CH₃, —OSO₂CF₃, —OSO₂C₄F₉), and also many other, less frequently used, groups (e.g. phosphonates, fluorosulfonates, diazonium salts).

Under the standard reaction conditions described in the literature, the coupling of halotoluenes with organoboron compounds often proceeds in only low yields, especially in the case of the economically important chloroaromatics. The use of tailored bases, solvents and catalyst systems makes it possible to carry out the reaction in satisfactory yields in some cases (J. Org. Chem. 1999, 64, 3804; J. Am. Chem. Soc. 1998, 120, 9722; Angew. Chem. 1998, 110(4), 492; DE-A-4326169).

The coupling of sterically hindered toluenes in which the leaving group is in the orthoposition relative to the methyl group has been found to be particularly problematical. In these cases, particularly when the leaving group is chlorine, an increased number of reactions which compete with C/C or C/N coupling become apparent, since the rate of the desired coupling reaction is greatly inhibited. Here too, the yield can be improved by use of tailored catalyst systems. However, the ligands used for the catalysis can usually be obtained only via a plurality of synthesis steps and are therefore expensive. In addition, they cannot be stored without problems or require large amounts of noble metal (Angew. Chem. Int. Ed. Engl. 1999, 27(24), 3387; J. Org. Chem. 1999, 64, 10; J. Am. Chem. Soc. 1998, 120, 7369).

### SUMMARY OF THE INVENTION

The novel process described here makes it possible to obtain substituted benzyl compounds and toluene derivatives in a simple manner and in high yields by means of Suzuki-type coupling reactions of an aromatic with an organoboron compound followed by a reduction step. The process is particularly suitable for preparing ortho-substituted benzyl compounds and toluene derivatives. The process can be applied to both intermolecular and intramolecular coupling reactions. Advantageously, only very small amounts of catalyst are required in the process.

### DESCRIPTION OF THE PREFERRED EMBODIMENTS

The invention provides a process for preparing compounds of the formula (IV),

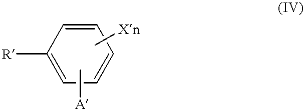

which comprises, in a process step 1, coupling an aromatic of the formula (II), bearing a group A and a leaving group LG located in the ortho, meta or para position relative thereto, with an organoboron compound of the formula (I) in the presence of a palladium and/or nickel catalyst and a base to form a compound of the formula (III),

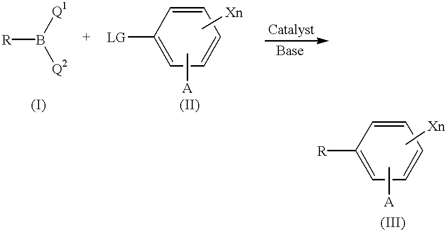

where

R is an unsubstituted or substituted aryl or heteroaryl radical, an unbranched or branched (C₁-C₁₈)-alkyl radical, an unsubstituted or substituted (C₂-C₈)-alkenyl radical or an unsubstituted or substituted (C₂-C₈)-alkynyl radical, where the aryl radical is preferably a (C₆-C₁₄)-aryl radical, in particular a phenyl, biphenyl or naphthyl radical, and the heteroaryl radical is preferably a 5- to 7-membered heteroaryl radical having from 1 to 3 N, S and/or O atoms, e.g. a pyridine, pyrimidine, pyrazine, pyridazine, 1,3-thiazole, 1,3,4-thiadiazole or thiophene radical and the substituents of the substituted aryl or heteroaryl radicals are preferably halogen, CN, OH, (C₁-C₄)-alkyl, (C₁-C₄)-alkoxy, NO₂, CF₃, CHO, NH₂, NHalkyl-(C₁-C₈), Nalkyl₂-(C₁-C₈), COOH, COO-alkyl-(C₁-C₈) or aryl, in particular phenyl,

preferably an unsubstituted or substituted (C₆-Ci₄)-aryl radical, an unbranched or branched (C₁-C₈)-alkyl radical, an unsubstituted or substituted (C₂-C₄)-alkenyl radical or an unsubstituted or substituted (C₂-C₄)-alkynyl radical and

in particular an unsubstituted or substituted phenyl or biphenyl radical,

Q¹, Q² are identical or different and are each OH, (C₁-C₄)-alkoxy, (C₁-C₄)-alkyl, phenyl or halogen, together form a (C₁-C₄)-alkylenedioxy group which may be substituted by from 1 to 4 (C₁-C₄)-alkyl groups, together form a 1,2-phenylenedioxy group or together with the boron atom are part of a boroxin ring of the formula (V), where the radicals R, independently of one another, are identical or different,

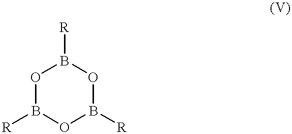

Q¹, Q² are preferably each OH or (C₁-C₄)-alkoxy, together form a (C₁-C₃)-alkylenedioxy group which may be substituted by from 1 to 4 methyl groups, or together with the boron atom are part of a boroxin ring of the formula (V), particularly preferably are each OH, butyl or isobutyloxy, together form an ethylenedioxy, 1,1,2,2-tetramethylethylenedioxy, propylene-1,3-dioxy or neopentyidioxy group or together with the boron atom are part of a boroxin ring of the formula (V),

A is a cyano group or a carbonyl function of the formula COR¹, where R¹ is H, an unbranched or branched (C₁-C₈)-alkyl radical or an unsubstituted or substituted aryl or heteroaryl radical,

preferably a cyano group or a carbonyl function of the formula COR¹ in which R¹ is H or an unbranched or branched (C₁-C₄)-alkyl radical, and in particular a cyano group, a formyl group or —COCH₃,

LG is a leaving group such as a halogen atom, a sulfonate group or a diazonium group,

preferably iodine, bromine, chlorine, —OSO₂CH₃ or —OSO₂CF₃, particularly preferably iodine, bromine or chlorine and in particular chlorine, and

X is hydrogen or a substituent selected from the group consisting of aryl, which may be unsubstituted or substituted by substituents preferably selected from the group consisting of halogen, CN, OH, (C₁-C₄)-alkyl, (C₁-C₈)-alkoxy, NO₂, CF₃, CHO, NH₂, NHalkyl-(C₁-C₈), Nalkyl₂-(C₁-C₈), COOH, COO-alkyl-(C₁-C₈) and aryl; (C₁-C₈)-alkyl, branched or unbranched, (C₁-C₈)-alkenyl, branched or unbranched, (C₁-C₈)-alkynyl, branched or unbranched, (C₁-C₈)-alkoxy,

(C₁-C₈)-acyloxy, Ophenyl, fluorine, chlorine, NO₂, NH₂, NHalkyl-(C₁-C₈), Nalkyl₂-(C₁-C₈), OH, CN, CHO, COOH, SO₃H, SO₃-alkyl-(C₁-C₈), SO₂NH₂, SO₂N(alkyl-(C₁-C₈))₂, SO₂-alkyl-(C₁-C₈), COO-alkyl-(C₁-C₈), CONH₂, CO-alkyl-(C₁-C₈), NHCHO, CF₃, 5-membered heteroaryl or 6-membered heteroaryl, or in each case two of the substituents X together form an aliphatic or aromatic 5- to 6-membered carbocyclic ring or heterocyclic ring, containing C, N, S and/or 0 atoms, where n can be 1, 2, 3 or 4 and X is preferably hydrogen or substituents selected from the group consisting of (C₁-C₄)-alkyl, (C₁-C₄)-alkoxy, fluorine, chlorine, NO₂, NH₂, NHalkyl-(C₁-C₈), Nalkyl₂-(C₁-C₈), OH, CN, CHO and/or COOH, where n can be 1, 2, 3 or 4,

and, in a process step 2, reducing the compound of the formula (III) to give the compound of the formula (IV)

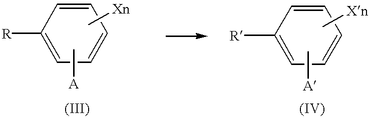

where

A′ is CH₃, CH₂NH₂, CH₂OH, CH(OH)R¹ or CH₂ R¹,

X′n is Xn or hydrogenated Xn and

R′ is R or hydrogenated R.

Organoboron compounds which are particularly preferred for the coupling reaction are compounds of the formula (V), where the radicals R, independently of one another, may be identical or different, and compounds of the formulae (VI), (VII) and (Vila),

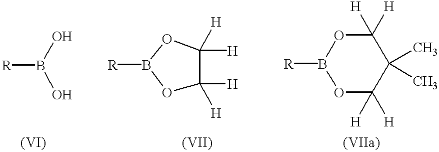

and mixtures thereof can be used.

If the organoboron compound (I) is part of the aromatic (II) bearing the leaving group LG, the coupling occurs as an intramolecular reaction.

Catalysts used for process step 1 are palladium metal, palladium compounds and/or nickel compounds. The catalysts can also have been applied to a solid support such as activated carbon or aluminum oxide.

Preference is given to palladium catalysts in which the palladium is present in the oxidation state (0) or (II), e.g. palladium ketonates, palladium acetylacetonates, nitrilepalladium halides, palladium halides, allylpalladium halides and/or palladium biscarboxylates.

Particular preference is given to palladium bisacetylacetonate, bis(benzonitrile)palladium dichloride, PdCl₂, Na₂PdCl₄, Na₂PdCl₆, bis(acetonitrile)palladium dichloride, palladium-II-acetate, bis(triphenylphosphine)palladium dichloride, tetrakis(triphenylphosphine)palladium, bis(diphenylphosphino)ferrocene-palladium dichloride and/or tetrachloropalladic acid.

The palladium compound can also be generated in situ, for example palladium(II) acetate from palladium(II) chloride and NaOAc.

The amount of catalyst is, based on the aromatic (II) bearing the leaving group LG, preferably from 0.001 to 0.5 mol % and particularly preferably from 0.01 to 0.2 mol %.

The catalyst can contain phosphorus-containing ligands or phosphorus-containing ligands can be added separately to the reaction mixture.

Preferred phosphorus-containing ligands are tri-n-alkylphosphines, triarylphosphines, dialkylarylphosphines, alkyldiarylphosphines and/or heteroarylphosphines such as tripyridylphosphine and trifurylphosphine, where the three substituents on the phosphorus can be identical or different and one or more substituents can link the phosphorus groups of two or more phosphines, with part of this linkage also being able to be a metal atom.

Particular preference is given to phosphines such as triphenylphosphine, tri-tert-butylphosphine, tricyclohexylphosphine, bis(diphenylphosphino)ferrocene and/or tris-(3-sulfophenyl)phosphine trisodium salt (TPPTS).

The total concentration of phosphorus-containing ligands is, based on the aromatic (II) bearing the leaving group LG, preferably up to 1 mol %, particularly preferably from 0.001 to 1 mol % and in particular from 0.01 to 0.5 mol %.

The bases usually used in process step 1 (coupling reaction) are alkali metal hydroxides, akaline earth metal hydroxides, alkali metal carbonates, alkaline earth metal carbonates, alkali metal hydrogencarbonates, alkali metal alkoxides, alkaline earth metal alkoxides, alkali metal fluorides, primary amines, secondary amines or tertiary amines.

Preference is given to bases such as sodium hydroxide, potassium hydroxide, lithium carbonate, sodium carbonate, potassium carbonate or potassium fluoride.

It is also possible to use mixtures of the bases. The amount of base used is preferably 1-10, particularly preferably 1-5 and in particular 1-2.5 mol-equivalents of base, based on the aromatic (II).

Solvents used for process step 1 are alcohols, polyols, polyethylene glycols, sulfoxides or mixtures thereof.

Preferred solvents are methanol, ethanol, butanol, isopropanol, ethylene glycol, glycerol, tetraethylene glycol, dimethyl sulfoxide or mixtures thereof. Particularly preferred solvents are ethylene glycol, methanol and dimethyl sulfoxide. In all cases, water, 1,2-dimethoxyethane, tetrahydrofuran or lipophilic solvents such as toluene, xylene, chlorobenzene or dichloromethane can be added as cosolvent.

To carry out process step 1, the starting materials, the solvent, the base, the catalyst and, if used, the ligand are advantageously mixed and reacted at a temperature of preferably from 0 to 200° C., particularly preferably 30-170° C. and in particular 50-150° C.

Apart from this single-vessel reaction, the reaction can also be carried out with the various reactants being metered in a controlled fashion during the reaction. In this case, various ways of metering-in the reactants are possible.

The molar ratio of aromatic (II) to organoboron compound (I) is preferably from 0.9 to 1.1.

In process step 2 of the process of the invention, the product from process step 1 is converted by reduction to, depending on the choice of reaction conditions, a benzyl derivative or a toluene derivative.

The reduction of aldehydes, ketones or nitriles of the formula (III) (A=CHO, COR¹ or CN) to give benzyl compounds of the formula (IV) (A′=CH₂OH, CHR¹OH or CH₂NH₂) or toluene derivatives of the formula (IV) (A′=CH₃, CH₂R¹) can be carried out by methods with which those skilled in the art are familiar. In the reduction, the groups R and Xn can be converted into the groups R′ and X′n, respectively. For example, nitro groups can be reduced to amino groups, alkenes can be reduced to alkanes, nitrites can be reduced to alkylamines and carbonyls can be reduced to alcohols.

The conversion of aldehydes of the formula (III) (A=CHO) into benzyl alcohols of the formula (IV) (A′=CH₂OH) can be carried out by means of:

Reductions using boron hydrides or aluminum hydrides, e.g. sodium borohydride, lithium aluminum hydride or sodium dihydrobis(2-methoxy-ethoxy)aluminate, in alcoholic solvents or ethers such as tetrahydrofuran or diethyl ether.

Catalytic hydrogenations using heterogeneous palladium, nickel or platinum catalysts, for example Pd on carbon, Raney nickel or platinum on carbon, and also homogeneous hydrogenation catalysts, for example tris(triphenylphosphine)rhodium(l) chloride, and elemental hydrogen in solvents such as alcohols (e.g. MeOH, EtOH), water or esters (e.g. butyl acetate). The reductions can be carried out at atmospheric pressure or under superatmospheric pressure.

The conversion of aldehydes of the formula (III) (A=CHO) into toluene derivates of the formula (IV) (A′=CH₃) can be carried out by means of: Catalytic hydrogenations using heterogeneous palladium or platinum catalysts, for example Pd on carbon or platinum on carbon, and elemental hydrogen in solvents such as alcohols (e.g. MeOH, EtOH), water or esters (e.g. butyl acetate), in the presence or absence of mineral acids such as sulfuric acid, phosphoric acid or hydrochloric acid, or in acetic acid. The reductions are preferably carried out under superatmospheric pressure and at temperatures of from 50 to 150° C. In the case of suitable substrates, hydrogenation without addition of solvent is also possible.

Transfer hydrogenations using heterogeneous palladium catalysts, e.g. palladium on carbon, and ammonium formate or formic acid as hydrogen source in suitable solvents, preferably formic acid.

Reductions using hydrazine by the Wolff-Kishner method (anhydrous hydrazine, sodium alkoxides) or the Huang-Minlon method (hydrazine hydrate, potassium hydroxide or sodium hydroxide).

Conversion of the aldehydes into the corresponding imines prior to the reduction may be advantageous.

The conversion of ketones of the formula (III) (A=COR¹) into benzyl alcohols of the formula (IV) (A′=CHR¹OH) can be carried out by means of:

Reductions using boron hydrides or aluminum hydrides, e.g. sodium borohydride, lithium aluminum hydride or sodium dihydrobis(2-methoxy-ethoxy)aluminate, in alcoholic solvents or ethers such as tetrahydrofuran or diethyl ether.

Catalytic hydrogenations using heterogeneous palladium, nickel or platinum catalysts, for example palladium, Raney nickel or platinum on carbon, and also homogeneous hydrogenation catalysts, for example tris(triphenylphosphine)rhodium(l) chloride, and elemental hydrogen in solvents such as alcohols (e.g. MeOH, EtOH), water or esters (e.g. butyl acetate). The reductions can be carried out at atmospheric pressure or under superatmospheric pressure.

The conversion of ketones of the formula (III) (A=COR¹) into toluene derivatives of the formula (IV) (A′=CH₂R¹) can be carried out by means of:

Clemmensen reductions using zinc and hydrochloric acid.

Wolff-Kishner or Huang-Minlon reductions using hydrazine in alkaline medium.

Catalytic hydrogenations using heterogeneous palladium or platinum catalysts, for example palladium or platinum on carbon, and elemental hydrogen in solvents such as alcohols (e.g. MeOH, EtOH), water or esters (e.g. butyl acetate), in the presence or absence of mineral acids such as sulfuric acid, phosphoric acid or hydrochloric acid, or in acetic acid.

The reductions are preferably carried out under superatmospheric pressure

and at temperatures of from 50 to 150° C. In the case of suitable substrates, hydrogenation without addition of solvent is also possible.

The conversion of nitriles of the formula (III) (A=CN) into benzylamines of the formula (IV) (A′=CH₂NH₂) can be carried out by means of:

Reductions using boron hydrides or aluminum hydrides, e.g. sodium borohydride, lithium aluminum hydride or sodium dihydrobis(2-methoxy-ethoxy)aluminate, in alcoholic solvents or ethers such as tetrahydrofuran or diethyl ether.

Catalytic hydrogenations using heterogeneous palladium, nickel or platinum catalysts, for example Pd on carbon, Raney nickel or platinum on carbon, and elemental hydrogen in solvents such as alcohols (e.g. MeOH, EtOH), water or esters (e.g. butyl acetate) or acetic acid, if desired under acidic conditions, for example by means of addition of hydrochloric acid, or under acylating conditions, for example in the presence of acetic anhydride or formic esters. The reductions can be carried out at atmospheric pressure or under superatmospheric pressure.

The conversion of nitriles of the formula (III) (A=CN) into toluene derivatives of the formula (IV) (A′CH₃) can be carried out by means of:

Catalytic hydrogenations using heterogeneous palladium, nickel or platinum catalysts, for example Pd on carbon, Raney nickel or platinum on carbon, and elemental hydrogen in solvents such as alcohols (e.g. MeOH, EtOH), water or esters (e.g. butyl acetate) or acetic acid, if desired under acidic conditions, for example by means of addition of hydrochloric acid, or under acylating conditions, for example in the presence of acetic anhydride or formic esters. The reductions can be carried out at atmospheric pressure or under superatmospheric pressure. Relatively high temperatures of from 80 to 150° C. are generally necessary.

Hydrogenations in the gas phase at temperatures above 250° C. in the presence of suitable catalysts, for example mixed nickel/copper catalysts or molybdenum sulfide.

With regard to the various methods of catalytic hydrogenation, reference may at this point be made to, for example, F. Zymalkowski, “Katalytische Hydrierungen im Organisch-Chemischen Laboratorium”, Ferdinand Enke Verlag, Stuttgart, 1965.

The products can be worked up by known methods with which those skilled in the art are familiar. For example, the product can be separated from the reaction mixture by extraction or precipitation and subsequently, depending on the product, worked up further by, for example, recrystallization, distillation, rectification, melt crystallization, sublimation or chromatography.

### EXAMPLES

### Example 1

15.5 g of 2-chloroacetophenone, 31.3 g of 4′-n-pentoxybiphenyl-4-boronic acid, 7.5 g of sodium carbonate and a mixture of 44 mg of palladium as a 22% strength aqueous chloride solution, 1 ml of water and 720 mg of a 0.6M aqueous TPPTS solution together with 120 ml of ethylene glycol and 16 ml of water are placed under nitrogen in a reaction vessel and heated to boiling for 4 hours. After cooling to room temperature, 150 ml of water are added, the mixture is stirred vigorously for another 20 minutes and the solid which remains is filtered off. Crystallization of the residue from acetone and drying at 50° C. under reduced pressure gives 32 g (89%) of 2-(4′-n-pentoxy[1,1′]biphenyl-4-yl)-acetophenone having a melting point of 86° C.

1 g of 2-(4′-n-pentoxy[1,1′]biphenyl-4-yl)acetophenone are hydrogenated at 115-120° C. and slightly superatmospheric hydrogen pressure in the presence of 0.3 g of 5% palladium on carbon in 30 ml of glacial acetic acid for 4 hours. After cooling to room temperature and addition of 30 ml of water, the solid which has precipitated is filtered off, dissolved in methanol, the catalyst is filtered off, the mother liquor is evaporated and the product which precipitates is dried overnight at 50° C. under reduced pressure. This gives 0.87 g (90%) of 2-ethyl-4″-n-pentoxy[1,1′:4′,1″]terphenyl having a melting point of 62-65° C.

### Example 2

15 g of 4-chloro-3-nitrobenzaldehyde, 13.2 g of 4-carboxyphenylboronic anhydride, 10 g of sodium carbonate and a mixture of 50 mg of palladium as 22% strength aqueous chloride solution, 1 ml of water and 820 mg of a 0.6 M aqueous TPPTS solution together with 145 ml of ethylene glycol and 10 ml of water are placed under nitrogen in a reaction vessel and heated to boiling for 4 hours. 200 ml of water are added and the mixture is acidified with concentrated hydrochloric acid to pH 1-2, which results in precipitation of the product. Crystallization from isopropanol and drying under reduced pressure gives 18.4 g (84%) of 4′-formyl-6′-nitrobiphenyl-4-carboxylic acid as a yellow solid having a melting point of 227-235° C.

2 g of 4′-formyl-6′-nitrobiphenyl-4-carboxylic acid, 827 mg of 5% palladium on carbon and a spatula tip of p-toluene sulfonic acid together with 100 ml of methanol are placed in a reaction vessel and hydrogenated by passing hydrogen through the mixture at room temperature for 6 hours. The mixture is heated to boiling, the catalyst is filtered off, the filtrate is cooled to 0° C. and the solid which precipitates is purified by boiling with ethanol. This gives 1.4 g (79%) of 2′-amino-4′-methyl-biphenyl-4-carboxylic acid having a melting point of 273-278° C.

### Example 3

15 g of 5-bromo-2-anisaldehyde, 9.1 g of o-tolylboronic anhydride, 500 mg of bis(triphenylphosphine)palladium(II)chloride and 22.2 g of potassium phosphate together with 120 ml of dimethoxyethane are placed in a reaction vessel and refluxed for 3 hours. After cooling to room temperature, 200 ml of water are added and the mixture is extracted three times with 200 ml each time of methylene chloride. Evaporation and chromatography of the residue on silica gel (heptane:methylene chloride 1:1) gives 13 g (82%) of 4-methoxy-2′-methylbiphenyl-3-carbaldehyde having a melting point of 66-69° C.

4 g of 4-methoxy-2′-methylbiphenyl-3-carbaldehyde, 2.66 g of 80% strength hydrazine hydrate solution and 4 g of potassium hydroxide together with 40 ml of triethylene glycol are placed in a reaction vessel and refluxed for 2 hours. The hydrazine hydrate/water mixture is then distilled off until a temperature at the bottom of 195° C. has been reached, and the residue is heated to boiling for a further 4 hours. After cooling to room temperature, 50 ml of water are added and the mixture is extracted three times with 40 ml each time of diethyl ether. Evaporation and distillation using a bulb tube gives 2.8 g (75%) of 2′,3-dimethyl-4-methoxy-biphenyl having a boiling point of 129° C. at 15 torr.

### Example 4

15 g of 2-chlorobenzonitrile, 14.8 g of p-tolueneboronic acid and 28.9 g of sodium carbonate together with 50 ml of p-xylene, 40 ml of DMSO and 10 ml of water are heated to 120° C. At 80° C., a mixture of 24.7 g of palladium acetate and 0.55 ml of 0.6 M aqueous TPPTS solution in 2.5 ml of DMSO is added. After the reaction is complete, the phases are separated. The aqueous phase is washed with 50 ml of xylene. The combined organic phases are washed with 20 ml of water and dried over sodium sulfate. The solvent is evaporated and the residue is crystallized from n-heptane. This gives 18.6 g (88%) of 4′-methylbiphenyl-2-carbonitrile having a melting point of 48-49° C.

50 g of 4′-methylbiphenyl-2-carbonitrile together with 2.5 g of 5% palladium on carbon and 125 ml of n-butanol are placed in a vessel and hydrogenated at 120° C. for 10 hours by passing hydrogen through the mixture. After filtering off the catalyst, the product solution is fractionated under reduced pressure to give 38.8 g (82%) of 2,4′-dimethylbiphenyl having a boiling point of 138-140° C. at 14 mbar.

### Example 5

466 g of 2-chloro-5-nitrobenzaldehyde, 298 g of phenylboronic anhydride, 186 g of sodium carbonate and a mixture of 1.04 g of palladium as 22% strength aqueous chloride solution, 24 ml of water and 17.1 g of a 0.6 M aqueous TPPTS solution together with 1600 ml of ethylene glycol and 200 ml of water are placed under nitrogen in a reaction vessel and heated to boiling for 5 hours. After cooling to room temperature, the solid is filtered off and washed with 750 ml of water. The crude product is suspended in 750 ml of isopropanol, admixed with 10 g of activated carbon, heated to boiling for 10 minutes and subsequently filtered hot. After cooling the mother liquor to 5° C., the product precipitates. This is filtered off, washed twice with 150 ml each time of cold isopropanol and dried at 45° C. under reduced pressure. This gives 488 g (86%) of 4-nitrobiphenyl-2-carbaldehyde having a melting point of 79.5° C.

68 g of 4-nitrobiphenyl-2-carbaldehyde and 44 g of aniline together with 300 ml of xylene are placed in a vessel fitted with a water separator and heated to boiling for 3 hours. After cooling to room temperature, 4 g of 5% palladium on carbon are added to the solution and the mixture is hydrogenated at 45 bar and 140° C. for 4 hours. After filtering off the catalyst, the product solution is fractionally distilled. This gives 49.9 g (91%) of 2-methylbiphenyl-4-amine having a boiling point of 175-179° C. at 12 torr.

1. A process for preparing compounds of the formula (IV) 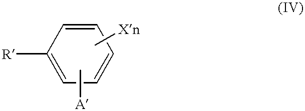
which comprises, in a process step 1, coupling an aromatic of the formula (II) bearing a group A and a leaving group LG located in the ortho, meta or para position relative thereto with an organoboron compound of the formula (I) in the presence of a palladium and/or nickel catalyst and a base to form a compound of the formula (III), 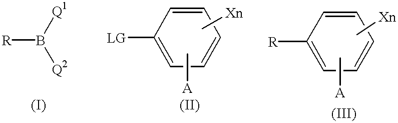
where
R is an unsubstituted or substituted aryl or heteroaryl radical, an unbranched or branched (C₁-C₁₈)-alkyl radical, an unsubstituted or substituted (C₂-C₈)-alkenyl radical or an unsubstituted or substituted (C₂-C₈)-alkynyl radical,
Q¹, Q² are identical or different and are each OH, (C₁-C₄)-alkoxy, (C₁-C₄)-alkyl, phenyl or halogen, together form a (C₁-C₄)-alkylenedioxy group, which may be unsubstituted or substituted by from 1 to 4 (C₁-C₄)-alkyl groups, or a 1,2-phenylene dioxy group or together with the boron atom are part of a boroxin ring of the formula (V), where the radicals R are identical or different, 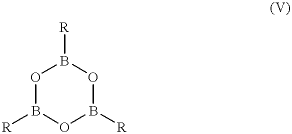
A is a cyano group or a carbonyl function of the formula COR¹, where R¹ is H, an unbranched or branched (C₁-C₈)-alkyl radical or an unsubstituted or substituted aryl or heteroaryl radical,
LG is a leaving group,
X is hydrogen or a substituent selected from the group consisting of aryl, substituted or unsubstituted, (C₁-C₈)-alkyl, branched or unbranched, (C₁-C₈)-alkenyl, branched or unbranched, (C₁-C₈)-alkynyl, branched or unbranched, (C₁-C₈)-alkoxy, (C₁-C₈)-acyloxy, Ophenyl, fluorine, chlorine, NO₂, NH₂, NHalkyl-(C₁-C₈), Nalkyl₂-(C₁-C₈), OH, CN, CHO, COOH, SO₃H, SO₃-alkyl-(C₁-C₈), SO₂NH₂, SO₂N(alkyl-(C₁-C₈))₂, SO₂-alkyl-(C₁-C₈), COO-alkyl-(C₁-C₈), CONH₂, CO-alkyl-(C₁-C₈), NHCHO, CF₃, 5-membered heteroaryl or
6-membered heteroaryl, or in each case two of the substituents X together form an aliphatic or aromatic 5-6-membered carbocyclic ring or heterocyclic ring containing C, N, S and/or 0 atoms, where n can be 1, 2, 3 or 4,
and, in a process step 2, reducing the compound of the formula (Ill) to give the compound of the formula (IV), where
A′ is CH₃, CH₂NH₂, CH₂OH, CH(OH)R¹ or CH₂ R¹,
X′n is Xn or hydrogenated Xn and
R′ is R or hydrogenated R.

2. The process as claimed in claim 1, wherein R is an unsubstituted or substituted (C₆-C₁₄)-aryl radical, an unbranched or branched (C₁-C₈)-alkyl radical, an unsubstituted or substituted (C₂-C₄)-alkenyl radical or a substituted or unsubstituted (C₂-C₄)-alkynyl radical.

3. The process as claimed in claim 1, wherein Q¹, Q² are identical or different and are each OH, butyl or isobutyloxy, together form an ethylenedioxy, 1,1,2,2-tetramethylethylenedioxy, propylene-1,3-dioxy or neopentyldioxy group or together with the boron atom are part of a boroxin ring of the formula (V).

4. The process as claimed in claim 1, wherein A is a cyano group or a carbonyl function of the formula COR¹, where R¹ is H or an unbranched or branched (C₁-C₄)-alkyl radical.

5. The process as claimed in claim 1, wherein LG is iodine, bromine, chlorine, —OSO₂CH₃ or —OSO₂CF₃.

6. The process as claimed in claim 1, wherein X is hydrogen, (C₁-C₄)-alkyl, (C₁-C₄)-alkoxy, fluorine, chlorine, NO₂, NH₂, NHalkyl-(C₁-C₈), Nalkyl₂-(C₁-C₈), OH, CN, CHO and/or COOH, and n can be 1, 2, 3 or 4.

7. The process as claimed in claim 1, wherein the palladium catalyst is palladium bisacetylacetonate, bis(benzonitrile)palladium dichloride, PdCl₂, Na₂PdCl₄, Na₂PdCl₆, bis(acetonitrile)palladium dichloride, palladium-II-acetate, bis(triphenylphosphine)palladium dichloride, tetrakis(triphenylphosphine)palladium, bis(diphenylphosphino)ferrocenepalladium dichloride and/or tetrachloropalladic acid.

8. The process as claimed in claim 1, wherein the amount of catalyst in process step 1, based on the aromatic (II) bearing the group LG, is from 0.001 to 0.5 mol %.

9. The process as claimed in claim 1, wherein the catalyst in process step 1 contains phosphorus-containing ligands selected from the group consisting of tri-n-alkylphosphines, triarylphosphines, dialkylarylphosphines, alkyldiarylphosphenes and heteroarylphosphines, where the three substituents on the phosphorus can be identical or different and one or more substituents can link the phosphorus groups of two or more phosphines, with part of this linkage also being able to be a metal atom.

10. The process as claimed in claim 9, wherein the phosphorus-containing ligands are triphenylphosphines, tri-tert-butylphosphine, tricyclohexylphosphine, bis(diphenylphosphino)ferrocene and/or tris-(3-sulfophenyl)phosphine trisodium salt.

11. The process as claimed in claim 9, wherein the total concentration of phosphorus-containing ligands, based on the aromatic (II) bearing the group LG, is from 0.001 to 1 mol %.

12. The process as claimed in claim 1, wherein the base in process step 1 is an alkali metal hydroxide, an alkaline earth metal hydroxide, an alkali metal carbonate, an alkaline earth metal carbonate, an alkali metal hydrogencarbonate, an alkali metal alkoxide, an alkaline earth metal alkoxide, an alkali metal fluoride, a primary amine, a secondary amine or a tertiary amine or a mixture thereof.

13. The process as claimed in claim 1, wherein process step 1 is carried out in the presence of water and/or a solvent selected from the group consisting of alcohols, polyols, polyethylene glycols, sulfoxides and mixtures thereof.

14. The process as claimed in claim 1, wherein process step 1 is carried out by mixing the starting materials, the solvent, the base, the catalyst and, if used, the ligand and reacting the mixture at a temperature of from 0 to 200° C.

15. The process as claimed in claim 1, wherein process step 2 is a catalytic hydrogenation using elemental hydrogen, a reduction using metal hydrides, a Clemmensen reduction, a Wolff-Kishner reduction, a Huang-Minlon reduction a nd/or a transfer hydrogenation.
